(19) United States
(12) Patent Application Publication
(10) Pub. No.: US 2016/0168524 A1
(43) Pub. Date: Jun. 16, 2016

(54) NOVEL METHOD FOR CULTIVATING MICRO-ORGANISMS BY CONFINEMENT IN MICRO-BIOREACTORS

(71) Applicants: ETABLISSEMENTS J. SOUFFLET, Nogent-sur-Seine (FR); ECOLE SUPERIEURE DE PHYSIQUE ET DE CHIMIE INDUSTRIELLES DE LA VILLE DE PARIS, Paris (FR)

(72) Inventors: Jairo Ivan GARNICA RODRIGUEZ, Paris (FR); Laurent BOITARD, Paris (FR); Antoine Serge Dominique DREVELLE, Romilly (FR); Jérôme BIBETTE, Paris (FR)

(73) Assignees: ETABLISSEMENTS J. SOUFFLET, Nogent-sur-Seine (FR); ECOLE SUPERIEURE DE PHYSIQUE ET DE CHIMIE INDUSTRIELLES DE LA VILLE DE PARIS, Paris (FR)

(21) Appl. No.: 14/903,938

(22) PCT Filed: Jul. 10, 2014
(86) PCT No.: PCT/EP2014/064800

§ 371 Date: Jan. 8, 2016

(30) Foreign Application Priority Data
Jul. 10, 2013 (FR) 1301631

(51) Int. Cl. C12M 3/06; C12M 1/12

(52) U.S. Cl. CPC C12M 23/16; C12M 25/01

(57) ABSTRACT

The present invention relates to a novel method for cultivating micro-organisms by confinement in micro-bioreactors. Said method comprises using a capillary tube in which a carrier fluid for moving a train of droplets forward flows, said capillary tube comprising micro-bioreactors in which the culture of said micro-organisms takes place, wherein said micro-bioreactors are separated by a spacing fluid which is a gas. The diameter of the micro-bioreactors (5) is smaller than that of the capillary tube (8) and the size of the bubble (6) of said spacing fluid is within a range of two to ten times the diameter of said capillary tube (8). The method can be used for cultivating micro-organisms such as thread-like fungi or planktonic algae.

The present invention is directed towards a novel method for the culture of microorganisms by confinement in micro-bioreactors.

More specifically, the present invention allows the kinetic monitoring over long incubation times (>24 h) of the culture of microorganisms in a confined medium.

It is known in particular from French patent No 11/00659 to conduct the culture of microorganisms in a confined medium; this application particularly describes a method and device for kinetic monitoring of a reaction with the confinement of microorganisms in aqueous reservoirs. This method is based on referencing of the sequence of aqueous reservoirs within a succession of aqueous reservoirs separated by a spacer fluid and carried by a carrier fluid in a tube. However, said method does not allow the examining of microorganisms having growth or biological activity that will modify the interfaces. In such cases, on and after 24 h, the integrity of the reservoirs is no longer preserved: either they merge together or they subdivide into smaller reservoirs. Interpretation of experimental results is no longer possible once at least one reservoir is lost, referencing also being lost, and/or when there is variation in size of one of said reservoirs e.g. through leakage; in this latter case, the concentration of metabolite that is internally measured will be artificially increased via a concentration effect and not because the microorganism contained in said reservoir is a super-producer of the metabolite under consideration.

As a general rule, existing technologies do not allow efficient confining of all types of microorganisms over times longer than 24 h.

It is the objective of the invention to be able to study an entire life cycle irrespective of types of microorganisms, their biological activities and growth rates.

More specifically, the method of the invention for the culture of microorganisms by confinement in micro-bioreactors is of the type comprising a capillary tube wherein there circulates a carrier fluid intended to cause the forward movement of a train of droplets, said micro-bioreactors being separated by a spacer fluid, this fluid being a gas.

According to one preferred embodiment of the invention, the diameter of the micro-bioreactors in which the culture of said microorganisms takes place is smaller than the diameter of said capillary tube.

Advantageously the size of the bubble of said spacer fluid is within a range of two to ten times the diameter of said capillary tube.

The method of the present invention will be better understood on reading the following description referring to the Figures that are given solely by way of indication and among which:

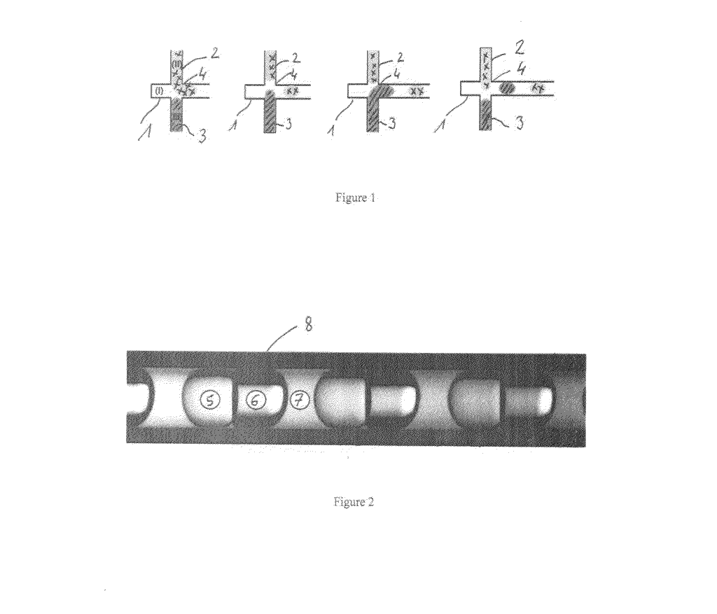

FIG. 1 schematically illustrates the composition of the droplet train in the state of the art;

FIG. 2 is a longitudinal section view of a capillary inside which there circulates a droplet train in the state of the art;

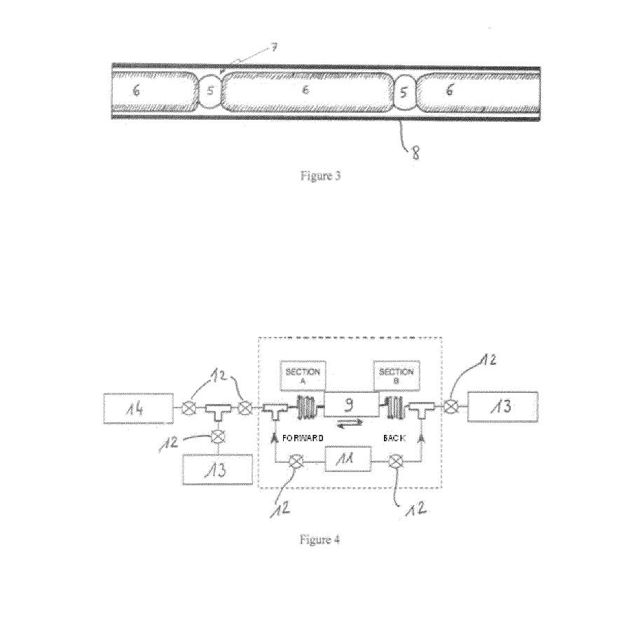

FIG. 3 is a longitudinal section view of a capillary inside which there circulates a droplet train according to the invention; and

FIG. 4 is a simplified illustration of the device assembly used in the method of the invention.

As illustrated in FIG. 1 the droplet train, as is usual, is formed of three mutually non-miscible phases (I), (II) and (III), each phase derived from a reservoir (not illustrated), valves (e.g. solenoid valves or air-operated valves, not illustrated) allowing the release of the different phases into their respective tube 1, 2 and 3 converging towards a crossway junction where said droplets are formed, one branch 4 of which is the capillary tube inside which the droplet train circulates. Phase (I) forms the carrier fluid, phase (II) forms the droplets in which the microorganisms are cultured and phase (III) forms the spacer fluid as will be specified further on in the present description. The size and spacing between the different droplets are dependent on the geometry of the junction and on the ratio between the injection flow rates of the phases. Finally the encapsulation of microorganisms inside the droplets of phase (11) follows Poisson's law.

The prior art, and in particular French patent No 11/00659, teaches how the previously described droplet train is formed; the present invention does not concern this method of obtaining said droplet train.

FIG. 2 provides a clearer view of part of the conventional droplet train formed of droplets 5 containing the reaction mixture in which the microorganisms will develop, separated from one another by droplets of spacer fluid 6 preventing the droplets 5 from merging together; the droplets 5 and 6 are carried forward by a carrier fluid 7 inside a capillary tube 8, said carrier fluid 7 allowing both movement of the droplets and lubrication of the capillary 8, preventing contamination between consecutive droplets 5. By capillary tube is meant a tube having an inner diameter smaller than 2 mm.

As previously mentioned, the droplet train is formed of three non-miscible phases. The carrier fluid (I) is most often a perfluorinated oil (“liquid Teflon”) not having any toxicity for the microorganism contained in the micro-bioreactor. To ensure proper formation of the train and to avoid any problem of contamination between droplets the carrier oil has higher affinity for the capillary than the other phases.

The second aqueous phase (II) contains the cells and the culture medium.

Finally, the third phase (III) does not mix with the two first; it may be formed of a liquid such as a hydrocarbon or mineral oil.

The formation of a droplet train in the state of the art allowing the culture of microorganisms in these droplets having been recalled, the present description will now focus on the improvements made to known methods thereby overcoming the shortcomings of said methods.

In the remainder of the present description, the droplets 5 in which the microorganisms are cultured will be called micro-bioreactors 5.

The method of the present invention can be applied to different microorganisms and in particular to filamentous fungi and planktonic algae.

Filamentous fungi form hydrophobic filaments (hyphae) capable of extending from one micro-bioreactor to another through the carrier liquid. If a liquid spacer composed of a hydrocarbon is used, the filaments will be able to pass completely therethrough as far as the neighbouring micro-bioreactor. They may also form biofilms at the micro-bioreactor/hydrocarbon interface which will gradually fully obstruct the cross-section of the capillary. This phenomenon leads to destruction of the train and end of the experiment.

To solve this problem it has been found—this being one of characteristics of the method of the invention—that it is possible to use a gas as spacer fluid. However, if the micro-bioreactor is too large and has a substantial contact surface area with the capillary, the phenomenon of obstruction due to development of the filamentous fungus may similarly occur. This gives rise to problems of contamination between the micro-bioreactors 5 and of droplet train stability due to interactions of the filaments with the capillary 8.

With regard to planktonic algae, on the edges of the droplet train a phenomenon of self-emulsification has been observed in aqueous micro-bioreactors 5 and in the spacer types of compartments. The most probable explanation is the presence of bacteria which coexist alongside the algae in the micro-bioreactors. These bacteria are capable of synthesizing surfactants thereby promoting self-emulsification and leading to collapse of the droplet train.

For planktonic algae, the use of a gas as spacer fluid is sufficient to solve the problem of self-emulsification.

However, with regard to filamentous fungi this replacement is insufficient to ensure stability of the train. The solution is then—this being another characteristic of the method of the invention—to reduce the size of the micro-bioreactor 5 so as to reduce interactions of the filaments with the walls of the capillary tube 8.

Among the different gases which can be used as spacer fluid, advantageous use is made of air first on account of its wetting properties and secondly because this provides conditions close to those of fermentation in a solid medium.

According to one variant of embodiment, a mixture of nitrogen/carbon dioxide is used as spacer fluid; this fluid is particularly advantageous when the microorganisms to be cultured are algae since this mixture promotes photosynthesis activity.

Irrespective of the spacer fluid used, which may be in the form of a gas mixture, said spacer fluid must:
be non-miscible with the carrier fluid and the content of the micro-bioreactors;
not interact with the microorganism i.e. the microorganism must not be able to grow or to propagate in the spacer fluid;
not be toxic i.e. harmful to growth of the microorganism.

According to one preferred embodiment of the method of the invention, the diameter of the micro-bioreactors 5 is smaller than the diameter of the capillary tube 8; more preferably, the diameter of the micro-bioreactors 5 lies within a range of between 80 and 85% of the diameter of the capillary tube 8. Said configuration is particularly advantageous when the microorganisms to be cultured in the micro-bioreactors 5 are filamentous fungi. Below a value of 80% there is a risk that successive air bubbles forming the spacer fluid 6 might come into contact underneath the micro-bioreactors 5 which will rapidly cause merging of spacer bubbles 6 and micro-bioreactors 5.

The droplet train used for growth of filamentous fungi is advantageously prepared in accordance with the following operating mode.

The spores of filamentous fungi are suspended in PGS medium (glucose 10 g/L, pancreatic peptone 6 g/L, MgSO₄ 7H₂O 0.5 g/L, KH₂PO4 0.5 g/L, FeSO₄ 7H₂O 0.5 mg/L, pH adjusted to 5). The carrier liquid is composed of Novec HFE-7500 fluorinated oil. The train is formed at a crossway junction of inner diameter 0.5 mm connected to a capillary tube in FEP 15 m in length and with an inner diameter of 0.75 mm. The PGS medium and HFE oil are injected by syringe pumps at respective flow rates of 5.0 and 3.5 mL/h. The air is injected via a solenoid valve at a pressure of 0.5 bar through a tube 50 cm in length and of inner diameter 0.2 mm. This tube allows sufficient hydrodynamic resistance to be set up to generate a homogeneous train. Air bubbles 10 cm in length are injected on each edge of the train allowing confining of the train.

In addition, it was observed with regard to the growth of algae and filamentous fungi that the spacer air bubbles 6 decrease over time due to biological activity inside the micro-bioreactors 5 (breathing and photosynthesis). As a result, if the spacer bubbles 6 are too small on initiating the method of the invention, there comes a time when some thereof disappear leading to coalescence of the micro-bioreactors 5 they had separated. Advantageously, to obtain a stable train for more than 90 h, the size of a spacer bubble 6 must be at least ten times larger than the inner diameter of the capillary tube 8.

The studied kinetics being of long duration, it was observed that edge effects occur. This results in a reduction in the size of the micro-bioreactors 5 at both edges of the train which can lead to coalescence of the micro-bioreactor 5 with its neighbour. This effect is also related to the fact that air is used as spacer fluid which promotes evaporation. To overcome this phenomenon, in one preferred embodiment of the method of the invention the carrier fluid reservoirs are saturated with water allowing recirculation of the train. One example of an embodiment of the method of the invention will now be described.
1/ Preparation of solutions:
   suspension of microorganisms in the culture medium having a concentration corresponding to the targeted occupation rate (number of cells/-micro-bioreactors)
   water-saturated perfluorinated oil with added perfluorinated surfactant (0.06%)
2/ Generation of the droplet train with:
   carrier fluid: HFE-7500 perfluorinated oil+0.06% surfactant
   spacer fluid: compressed air
   reactants: suspensions of microorganisms
The ratio of the flow rates/pressures of the three fluids is adjusted to obtain:
a) size of micro-bioreactor 5 between 80 and 85% the inner diameter of the tube 8;
b) size of spacer bubble 6 at least three times the inner diameter of the tube 8.
The size characteristics given in a) and b) above are shown in FIG. 3.
3/ Adding terminal plugs of spacer fluid in the form of a long bubble 6 at the start and end of the droplet train.
4/ Quality control of the droplet train (80%<size of micro-bioreactors<85%), via image analysis.
5/ Kinetic monitoring via forward-and-back movement of the droplet train in front of the detector 9.
6/ Analysis of data recorded for each micro-bioreactor.

By way of indication the Table below groups together the different parameters (spacer fluid, size of micro-bioreactors or of spacer fluid bubbles) and gives the maximum incubation time of microorganisms as a function of these parameters.

| | Size of micro- | | Size of drops or | |
|---|---|---|---|---|
| | bioreactor (as % | | bubbles of spacer | Maximum |
| Test | of inner diameter | Spacer | fluid (as % of inner | incubation |
| No | of capillary tube) | fluid | diameter of the tube) | time |
| 1 | 100% | Mineral | 200% | 48 h |
| | | oil | | |
| 2 | 100% | Mineral | 200% | 34 h |
| | | oil | | |
| 3 | 100% | Mineral | 200% | 28 h |
| | | oil | | |
| 4 | 100% | PDMS | 200% | 13 h |
| | | (100 cSt) | | |
| 5 | 100% | PDMS | 200% | 20 h |
| | | (50 cSt) | | |
| 6 | 100% | Squalene | 200% | 25 h |
| 7 | 100% | Air | 1000% | 22 h |
| 8 | 95% | Air | 1000% | 30 h |
| 9 | 85% | Air | 1000% | 91 h |
| 10 | 85% | Air | 600% | 60 h |
| 11 | 90% | Air | 1200% | 68 h |
| 12 | 90% | Air | 1200% | 162 h |
| 13 | 90% | Air | 1500% | 109 h |

In the above Table the following microorganisms were cultured:
Aspergillus Oryzae (filamentous fungus) for tests 1-7 and 11;
Aspergillus Niger (filamentous fungus) for tests 8-10;
Chlamydomonas Reinhardti (single-cell alga) for tests 12-13.

With regard to the aforementioned filamentous fungi, the best results in terms of stability were obtained in tests 9, 10 and 11.

By means of the improvements brought by the method of the invention, it was able to be demonstrated that it is possible to monitor the growth and biological activity kinetics in micro-bioreactors over time lengths hitherto inaccessible to the person skilled in the art.

Although algae and yeasts do not pass through the carrier oil/culture medium barrier during their growth, this confinement method results in increased stability of the droplet train reducing the interaction between the droplet of culture medium and the walls of the tube, bearing in mind that a decrease in surface tension due to the metabolites secreted by the microorganisms during their growth affects the stability of the train over time. Finally, the use of gas bubbles can allow the regulating of gas exchanges by acting as reservoir of oxygen (breathing of filamentous fungi) or of carbon dioxide (photosynthesis of algae).

The micro-bioreactors 5 are arranged in a unidimensional train which may vary by several hundred to several thousand samples. Each micro-bioreactor 5 is identified by its rank in the train. The integrity of the train of micro-bioreactors is therefore essential to ensure reactions over long time periods. The micro-bioreactors 5 are continually set in movement to preserve the lubrication film and cause homogenization of the micro-bioreactor via recirculation. By passing the train in front of a detector 9 such as illustrated in FIG. 3 in one direction and then in the other, it is possible to monitor the reactions inside each micro-bioreactor 5 over time. It is also possible to pass the train of micro-bioreactors 5 in front of the detector always in the same direction ensuring a recirculation loop, allowing the monitoring over time of the reactions taking place inside each micro-bioreactor.

This detector 9 is integrated in an incubation module 10 comprising in particular a pump 11 and valves 12 (solenoid valves or air-operated valves for example) allowing the train of micro-bioreactors to circulate in one direction an then in the other, the train being loaded at section A then moved in front of the detector 9 towards section B. The presence of outlets 13 allows the elimination of undesirable micro-bioreactors 5 and cleaning of the circuit once the experiment is terminated. A module 14 completes the present system, a module in which the droplet train is formed (micro-bioreactors and bubbles of fluid) conforming to FIG. 1 with the different reservoirs containing phases (I), (II) and (III).

1) A method for the culture of microorganisms by confinement in micro-bioreactors (5) of the type comprising a carrier fluid intended to move forward, inside a capillary tube (8), a train of droplets formed of micro-bioreactors (5) in which there takes place the culture of microorganisms able to modify the interfaces of said droplets, wherein said micro-bioreactors (5) are separated by a spacer fluid, and wherein said spacer fluid is a gas.

2) The method according to claim 1 wherein said gas is air.

3) The method according to claim 1 wherein said gas is a mixture of nitrogen and carbon dioxide.

4) The method according to claim 1 wherein the diameter of said micro-bioreactors (5) is smaller than the diameter of said capillary tube (8).

5) The method according to claim 4 wherein said diameter of said micro-bioreactors (5) is between 80% and 85% of the diameter of said capillary tube (8).

6) The method according to claim 1 wherein said spacer fluid is in the form of a bubble (6) having a size in the range of two to ten times the diameter of said capillary tube (8).

7) The method according to claim 1 wherein said carrier fluid is derived from a water-saturated reservoir.

8) The method according to claim 1 wherein the cultured microorganism is a filamentous fungus.

9) The method according to claim 8 wherein said filamentous fungus is Aspergillus Oryzae or Aspergillus Niger.

10) The method according to claim 1 wherein the cultured microorganism is a planktonic alga.

11) The method according to claim 10 wherein said alga is Chlamydomonas Reinhardti.
